(19) United States
(12) Patent Application Publication
(10) Pub. No.: US 2025/0223658 A1
(43) Pub. Date: Jul. 10, 2025

(54) SAMPLE EXTRACTION TUBE FOR METHOD FOR DETECTION OF RNA OR DNA

(71) Applicant: STEM ARTS PROJECTS, LLC, LINCOLN, NE (US)

(72) Inventors: Abraham Oommen, Lincoln, NE (US); Govardhan Rathnaiah, Lincoln, NE (US); Alicia Dietrich, Lincoln, NE (US); Yashitola Wamboldt, Lincoln, NE (US); Matthew Greenleaf, Lincoln, NE (US); Heather Piscatelli, Lincoln, NE (US); Alyssa Hangman, Lincoln, NE (US)

(21) Appl. No.: 18/989,591

(22) Filed: Dec. 20, 2024

Related U.S. Application Data
(63) Continuation of application No. PCT/US23/25743, filed on Jun. 20, 2023.
(60) Provisional application No. 63/354,162, filed on Jun. 21, 2022.

(51) Int. Cl. C12Q 1/70; C12Q 1/6806; C12Q 1/6844; C12Q 1/6883

(52) U.S. Cl. CPC C12Q 1/701; C12Q 1/6844; C12Q 1/6883; C12Q 1/6806

(57) ABSTRACT

A method of detection of nucleic acids from a biological sample (molecular diagnostics) using a sample extraction tube without isolation or purification of the nucleic acids or the use of specialized equipment in the preparation of the biological sample is described. The method may include direct detection of nucleic acids from a biological sample without isolating or purifying nucleic acids (i.e. without isolation or purification of nucleic acids from other cellular components through centrifuges or magnetic beads) prior to analysis or the use of specialized equipment in the sample preparation and the PCR amplification (i.e. pipettes, PCR cartridges, or centrifuges).

This application claims the benefit of priority to U.S. Provisional Patent Application No. 63/354,162 filed Jun. 21, 2022, entitled “SAMPLE EXTRACTION TUBE FOR METHOD FOR DETECTION OF RNA OR DNA” which is incorporated by reference in its entirety; and is filed as a continuation of and claiming priority to PCT/US23/25743 filed Jun. 20, 2023, entitled “SAMPLE EXTRACTION TUBE FOR METHOD FOR DETECTION OF RNA OR DNA” which is incorporated by reference in its entirety. This application includes the material in the Sequence Listing XML file, entitled M1864-00142US created on Mar. 31, 2025, having a size of 7 KB, which is incorporated by reference in its entirety.

### BACKGROUND

The rapid detection of nucleic acids (i.e. deoxyribose nucleic acid (DNA) or ribonucleic acid (RNA)) in biological samples are a significant need. Presently, due to the increase in pandemic situations (e.g. Covid-19 pandemic), rapid identification of pathogens including virus, bacteria and fungi are in high demand, and rapid detection of nucleic acids from an organism's own genome remains of high interest. Many conventional methods available for the detection of nucleic acids require purification or isolation (i.e. separation of the nucleic acids from other cellular components for analysis through centrifuges or magnetic beads) of the nucleic acids prior to detection or identification of nucleic acids in an assay. For example, conventional PCR (polymerase chain reaction) techniques include the use of centrifuges or magnetic beads to isolate nucleic acids from other cellular components and debris prior to amplification and detection. Further, conventional sample collection and preparation includes pipetting the biological sample and/or PCR reagents (including PCR isolation reagents) into conventional PCR tubes to prepare the sample for PCR analysis.

Other conventional real-time PCR protocols include the use of cartridges, where the sample is placed in the cartridge containing all necessary reagents for nucleic acid purification and amplification. The cartridge maintains these reagents separately and delivers the reagents to the sample when needed, such as through pneumatic fluidics. These conventional PCR techniques include the use of specialized equipment for sample preparation and amplification (i.e. pipettes, centrifuges, cartridges separating the reagents from the sample with delivery occurring through pneumatic fluidics), which requires specialized training for proper use.

Conventional diagnostic antigen tests may utilize sample extraction tubes for the direct detection (i.e. the sample does not go through a process where the antigen is isolated from all other cellular components) of antigens such as SARS-COV2, streptococci that causes strep A, and proteins, such as human chorionic gonadotropin for early detection of pregnancy. These sample extraction tubes are made from a flexible plastic material (i.e. the sample extraction tube may be manipulated (squeezed together) by hand and retains its original shape when the manipulation ceases), such as polyethylene or polypropylene. Sample extraction tubes are typically a length from 72 to 80 millimeters (mm), and are generally cylindrical in shape with a diameter from 7 to 9 mm, with the sample extraction tube having a maximum volume capacity from 2-5 milliliters (mL). Sample extraction tubes may include a cap, such as a screw cap, for retaining the contents of the tube. The cap may further include a dropper opening to allow contents to leave the tube (e.g. the tube is squeezed, where the tube and cap function as a dropper).

A conventional method of antigen detection using the sample extraction tube is shown in FIG. 1. In 102, the sample extraction tube is opened and filled with a buffer configured for dilution of the sample to prepare it for detection. In 104, the sample is obtained, such as through a nasal swab using a conventional cotton tipped or flocked nylon swab. In 106, the sample collected on the swab is inserted into the sample extraction tube and mixed with the buffer. In 108, the swab is removed from the sample extraction tube, where during removal the sample extraction tube is squeezed to contact the swab to assist the sample collected on the swab to transfer to the buffer. In 110, the cap is inserted onto the sample extraction tube. In 112, the buffer containing the diluted sample is delivered to a lateral flow detection device using the cap and sample extraction tube as a dropper. In 114, a requisite amount of time for development and detection of the antigen on the lateral flow is allowed. In 116, the lateral flow device is read for positive or negative detection of the antigen. Direct antigen detection is advantageous as it does not utilize specialized equipment in the preparation of the biological sample, nor does it require the separation of the antigen from other cellular components prior to detection.

Therefore, it is desirable for a sample extraction tube to be used for a method of nucleic acid detection that does not require isolation or purification of the nucleic acids prior to detection or identification of the nucleic acid. It is further desirable to perform the method of nucleic acid detection that utilizes a sample extraction tube and does not utilize specialized equipment in the preparation of the sample. It is further desirable for a method of nucleic acid detection to eliminate the use of viral transport media or a universal transport media.

### SUMMARY

In aspects of the invention, the method of direct human, animal, microbial, and viral nucleic acid detection from a collected biological sample using a sample extraction tube without isolation or purification of the nucleic acids and without the use of specialized equipment in the collected biological sample preparation, the method including transferring the collected biological sample to a sample extraction tube having a treatment buffer configured for stabilizing the nucleic acids of the collected biological sample, the sample extraction tube including a tube configured for accommodating a volume of liquid from 0.5 to 5 milliliters, the tube made of a flexible plastic material; a cap configured for removable attachment to the tube, the cap made of a plastic material that is rigid, wherein the cap has a dropper opening and a dropper cap; heating the collected biological sample transferred to the sample extraction tube from 2 to 10 minutes at from 80 to 95 degrees Celsius; dispensing the collected biological sample from the dropper opening of the sample extraction tube to at least one reaction vessel; analyzing the collected biological sample for the nucleic acid; reporting results of the analysis to determine the presence or absence of the nucleic acid.

In aspects, the method of paragraph [0006], wherein the nucleic acid analyzed is a nucleic acid of an organism's genome.

In aspects, the method of paragraph [0006], wherein the nucleic acid analyzed is a nucleic acid from a pathogen.

In aspects, the method of paragraph [0006], wherein the analyzing is a nucleic acid amplification reaction selected from the group consisting of polymerase chain reaction (PCR), reverse transcriptase (RT) PCR, real-time PCR, real-time quantitative PCR, and isothermal amplification.

In aspects, the method of paragraph [0006], wherein the collected biological sample is a nasal swab from a human, where the nucleic acid analyzed detects the presence of any RNA or DNA virus.

In aspects, the method of paragraph [0010], wherein the collected biological sample is a nasal swab from a human, where the nucleic acid analyzed is the RdRp gene of SARS-COV2; the treatment buffer is 0.125 mM sodium citrate of pH 6.62, 1 mM TCEP (tris(2-carboxyethyl) phosphine) of pH 4.5, and 0.04 mg/mL PVSA (polyvinyl sulfonic acid), and wherein the analysis is real-time polymerase chain reaction with fluorescence detection.

In aspects, the method of paragraph [0006], wherein the collected biological sample is a nasal swab from a human, where the nucleic acid analyzed detects the presences of nucleic acids that cause hereditary genetic conditions in humans.

In aspects, the method of paragraph [0012], wherein the treatment buffer is 0.025 mM Sodium Citrate of pH 6.62, 0.2 mM TCEP (tris(2-carboxyethyl)phosphine) of pH 4.5, and 0.008 mg/mL polyvinyl sulfonic acid (PVSA), and wherein the analysis is polymerase chain reaction with agarose gel electrophoresis.

In aspects, the method of paragraph [0012], wherein the analysis is real-time polymerase chain reaction with fluorescence detection.

In aspects, the method of paragraph [0012], wherein the treatment buffer is 0.025 mM

Sodium Citrate of pH 6.62, 0.2 mM TCEP (tris(2-carboxyethyl) phosphine) of pH 4.5, and 0.008 mg/mL polyvinyl sulfonic acid (PVSA), and wherein the analysis is isothermal amplification with fluorescence detection.

In aspects of the invention, a method of direct human, animal, microbial, and viral nucleic acid detection from a collected biological sample using a sample extraction tube, the method including transferring the collected biological sample to the sample extraction tube without prior isolation or purification of nucleic acids having a treatment buffer configured for stabilizing the nucleic acids of the collected biological sample, the sample extraction tube comprising a tube configured for accommodating a volume of liquid from 0.5 to 5 milliliters, the tube made of a flexible plastic material; a cap configured for removable attachment to the tube, the cap made of a plastic material that is rigid, wherein the cap has a dropper opening; heating the collected biological sample transferred to the sample extraction tube from 2 to 10 minutes at from 80 to 95 degrees Celsius; dispensing the collected biological sample from the dropper opening of the sample extraction tube to at least one reaction vessel without the use of specialized equipment; analyzing the collected biological sample for the nucleic acid without isolation or purification of the nucleic acids prior to the analyzing; reporting results of the analysis to determine the presence or absence of the nucleic acid.

In aspects of the invention, the method of paragraph [0016], wherein the sample extraction tube further comprises a dropper cap.

In aspects of the invention, the method of paragraph [0016], wherein the treatment buffer comprises a buffering agent.

In aspects of the invention, the method of paragraph [0018], wherein the buffering agent is selected from the group consisting of phosphate buffered saline, from 5 to 50 milli-Molar (mM) Tris HCl, from 0.05-0.5 mM sodium citrate of pH from 6.0 to 7.0, 3 mM magnesium chloride, and 75 mM potassium chloride, and combinations thereof.

In aspects of the invention, the method of paragraph [0018], wherein the treatment buffer further comprises a chelating agent that stabilizes the released nucleic acids of the biological sample and interacts with other cellular components and cellular debris contained in the sample to facilitate analyzing the nucleic acids without isolation or purification of the nucleic acids.

In aspects of the invention, the method of paragraph [0015], wherein the buffering agent is selected from the group consisting of phosphate buffered saline, from 5 to 50 milli-Molar (mM) Tris HCl, from 0.05-0.5 mM sodium citrate of pH from 6.0 to 7.0, 3 mM magnesium chloride, and 75 mM potassium chloride, and combinations thereof; and the chelating agent is selected from the group consisting of from 0.1 to 1 milliMolar (mM) EDTA of pH 8.0, 2 mM DCTA of pH 8, from 0.5-4 mM DTPA of pH 8, from 0.25 to 5 mM TCEP-HCl of pH 4.5, 1 mM EGTA, and from 5% to 10% (weight/volume) of Bovine Serum Albumin, and combinations thereof.

In aspects of the invention, the method of paragraph [0020], wherein the treatment buffer further comprises a lysis agent that further facilitates lysis of cellular membranes, nuclei, and protein coats.

In aspects of the invention, the method of paragraph [0022], wherein the buffering agent is selected from the group consisting of phosphate buffered saline, from 5 to 50 milli-Molar (mM) Tris HCl, from 0.05-0.5 mM sodium citrate of pH from 6.0 to 7.0, 3 mM magnesium chloride, and 75 mM potassium chloride, and combinations thereof;

the chelating agent is selected from the group consisting of from 0.1 to 1 milliMolar (mM) EDTA of pH 8.0, 2 mM DCTA of pH 8, from 0.5-4 mM DTPA of pH 8, from 0.25 to 5 mM TCEP-HCl pH 4.5, 1 mM EGTA, and from 5% to 10% (weight/volume) of Bovine Serum Albumin, and combinations thereof; and the lysis agent is selected from the group consisting of from 20 to 250 mM guanidine isothicynate and 2 mM TCEP-HCl, and combinations thereof.

In aspects of the invention, the method of paragraph [0022], wherein the treatment buffer further includes an RNase inhibitor.

### FIGURES

FIG. 1 represents a conventional method of antigen detection using a sample extraction tube.

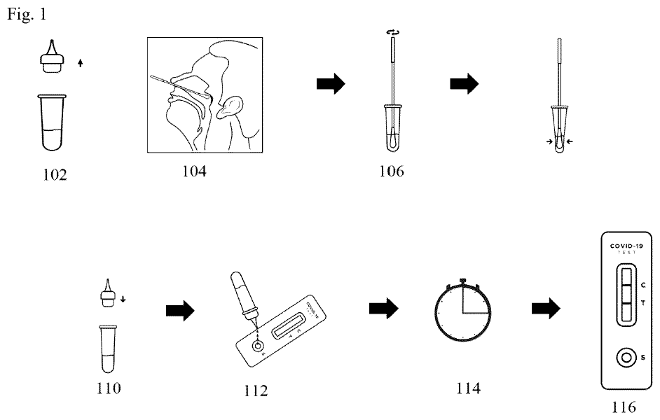

FIGS. 2a, 2b, and 2c represent a sample extraction tube having a treatment buffer (FIG. 2c) for use in a method for direct nucleic acid detection from a biological sample without isolation or purification of nucleic acids prior to detection or identification of the nucleic acids and without the use of specialized equipment in the preparation of the collected biological sample.

FIG. 3 represents a method for nucleic acid detection from a biological sample with a sample extraction tube using the treatment buffer and without isolation or purification of nucleic acids prior to detection or identification of the nucleic acids and without the use of specialized equipment in the preparation of the collected biological sample.

FIG. 4 is a first pictorial representation of the method using the sample extraction tube for nucleic acid detection from a collected biological sample without isolation or purification of nucleic acids prior to detection or identification and without the use of specialized equipment in the preparation of the collected biological sample for amplification.

FIG. 5 is a second pictorial representation of the method using the sample extraction tube for nucleic acid detection from a collected biological sample without isolation or purification of nucleic acids prior to detection or identification and without the use of specialized equipment in the preparation of the collected biological sample for amplification using a limited well thermal cycling device.

FIG. 6 demonstrates the efficacy of the method 300 in detecting nucleic acids of a human organism's genome.

FIG. 7 demonstrates the efficacy of the method 300 in detecting SARS-COV2 using a limited well thermal cycling device.

FIG. 8 demonstrates the efficacy of the method 300 in detecting SARS-COV2 using a conventional real-time PCR device.

### DETAILED DESCRIPTION

A method of detection of nucleic acids from a biological sample (molecular diagnostics) using a sample extraction tube without isolation or purification of the nucleic acids or the use of specialized equipment in the preparation of the biological sample is described. The method may include direct detection of nucleic acids from a biological sample without isolating or purifying nucleic acids (i.e. without isolation or purification of nucleic acids from other cellular components through centrifuges or magnetic beads) prior to analysis or the use of specialized equipment (i.e. pipettes, PCR cartridges, or centrifuges) in the sample preparation and the PCR amplification. The method may include collection of the biological sample directly into the treatment buffer without the use of a viral transport media (VTM) or universal transport media (UTM). Biological samples may be blood, urine, tissue, swabs (nasal, buccal, ocular, vaginal or anal). The nucleic acids for detection may be nucleic acids present in the organism's genome (including mutations of genes present in an organism) or nucleic acids from a pathogen.

The following terms have their assigned meaning as used in the application:
EDTA means ethylene diamine tetra acetic acid
EGTA means ethylene glycol-bis(β-aminoethyl ether)-N,N,N′,N′-tetraacetic acid
DCTA means 1,2-Cyclohexanedinitrilotetraacetic acid
DTPA means diethylenetriaminepentaacetic acid
TCEP-HCl means Tris(2-carboxyethyl)phosphine hydrochloride
Tris HCl means Tris(hydroxymethyl)aminomethane hydrochloride

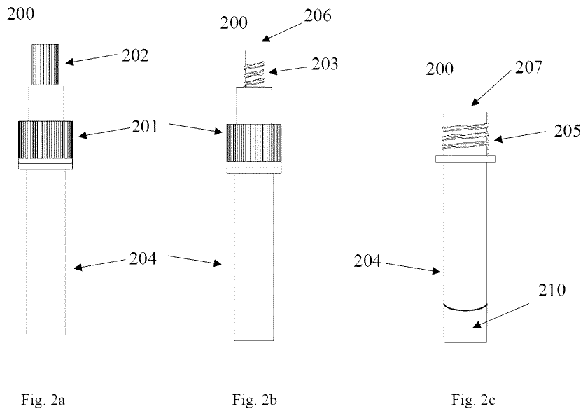

FIGS. 2a, 2b, and 2c represent a sample extraction tube having a treatment buffer (FIG. 2c) for use in a method for nucleic acid detection from a biological sample without isolation or purification of nucleic acids prior to detection and without the use of specialized equipment in the collected biological sample preparation. The sample extraction tube 200 may be a conventional sample extraction tube and includes a tube 204 and may include a cap 201.

The tube 204 of the sample extraction tube is made from a flexible plastic material (i.e. the sample extraction tube may be manipulated (squeezed together) by hand but retains its original shape when the manipulation ceases), such as polyethylene or polypropylene. The tube 204 may be a length from 72 to 80 millimeters (mm), and is generally cylindrical in shape with a diameter from 7 to 9 mm, with the tube having a maximum volume capacity from 2-5 milliliters (mL).

The cap 201 of the sample extraction tube 200 is configured for removable attachment to the tube 204 such as through threaded screws of the tube (represented by 205), a snapping cap, or the like. When removably attached to the tube 204, the cap 201 retains the contents of the tube 204 from exiting the tube opening 207 when the tube 204 is inverted. The cap is made of a plastic material that is rigid (i.e. less flexible than the tube 204).

The cap 201 may further include a dropper opening 206 that allows the contents of the tube 204 to leave the tube (e.g. the tube 204 is squeezed causing the tube 204 and cap 201 to act as a dropper), as shown in FIG. 2b. The cap 201 may further include a dropper cap 202 configured for removable attachment to the cap 201 such as through threaded screws of the cap (represented by 203), a snapping cap, or the like. When removably attached to the cap 201, the dropper cap 202 further retains the contents of the tube from exiting the dropper opening 206, when the sample extraction tube 200 is inverted, as shown in FIG. 2a.

FIG. 2c represents the tube 204 of the sample extraction tube 200 having a treatment buffer 210 for use in a method for nucleic acid detection from a biological sample using the sample extraction tube without isolation or purification of nucleic acids prior to detection or identification of the nucleic acid and without the use of specialized equipment in the collected biological sample preparation. The treatment buffer is configured for stabilizing the collected biological sample, where the treatment buffer is specific to the collected biological sample. The treatment buffer includes at least one buffer agent. The treatment buffer may further include a chelating agent, a lysis agent, and/or an RNase inhibitor.

The at least one buffering agent of the treatment buffer stabilizes the biological sample by maintaining a pH of the treatment buffer contacted biological sample from between pH 5 to 9. The buffering agent may be phosphate buffered saline, from 5 to 50 milli-Molar (mM) Tris HCl, from 0.05-0.5 mM sodium citrate of pH from 6.0 to 7.0, 3 mM magnesium chloride (MgCl₂, and 75 mM potassium chloride (KCl), and combinations thereof.

The chelating agent of the treatment buffer stabilizes the released nucleic acids of the biological sample and interacts with other cellular components and cellular debris contained in the sample to facilitate analyzing the nucleic acids without isolation or purification of the nucleic acids. The chelating agent may be from 0.1 to 1 milliMolar (mM) EDTA of pH 8.0, 2 mM DCTA of pH 8, from 0.5-4 mM DTPA of pH 8, from 0.25 to 5 mM TCEP-HCl of pH 4.5, 1 mM EGTA, and from 5% to 10% (weight/volume) of Bovine Serum Albumin, and combinations thereof.

The treatment buffer may further include a lysis agent to further facilitate lysis of the cellular membrane, nucleus, and protein coat in the case of a virus to further facilitate release of the nucleic acids of the biological sample into the treatment buffer. The lysis agent includes from 20 to 250 mM guanidine isothicynate and 2 mM TCEP-HCl and combinations thereof.

The treatment buffer may further include an RNase inhibitor to prevent degradation of RNA in the collected biological sample. The RNase inhibitor may include 0.02 milligrams (mg)/mL to 2.0 mg/mL PVSA (polyvinyl sulfonic acid).

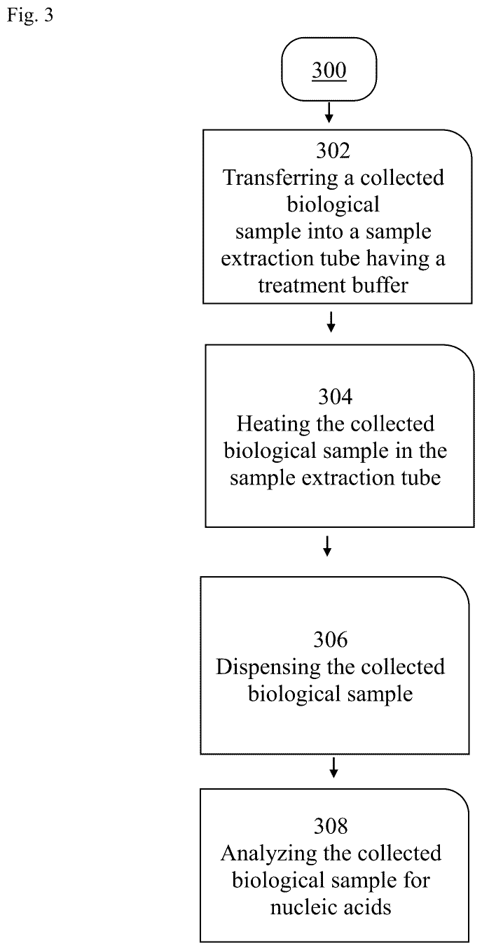

FIG. 3 represents a method for direct nucleic acid detection from a biological sample using a sample extraction tube and a treatment buffer and without isolation or purification of nucleic acids prior to detection and without the use of specialized equipment in the preparation of the collected biological sample. In 302, a collected biological sample is transferred into a sample extraction tube having a treatment buffer. For example, the collected biological sample may be a nasal swab, nasopharyngeal swab, buccal swab, throat swab, or urogenital or anal swab that are collected using a cotton tipped swab or flocked nylon swab. Or for example, the collected biological sample may be a biological sample in a viral transport media (VTM) or universal transport media (UTM) to prevent degradation of the nucleic acids when detection will not occur simultaneous to collection. Or for example, the collected biological sample may be blood collected on a glass rod.

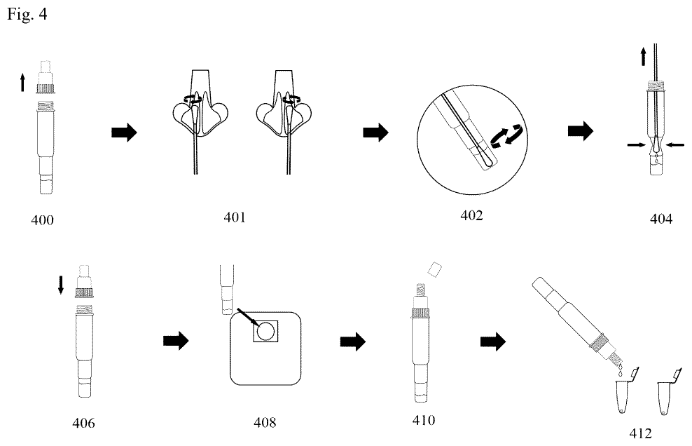

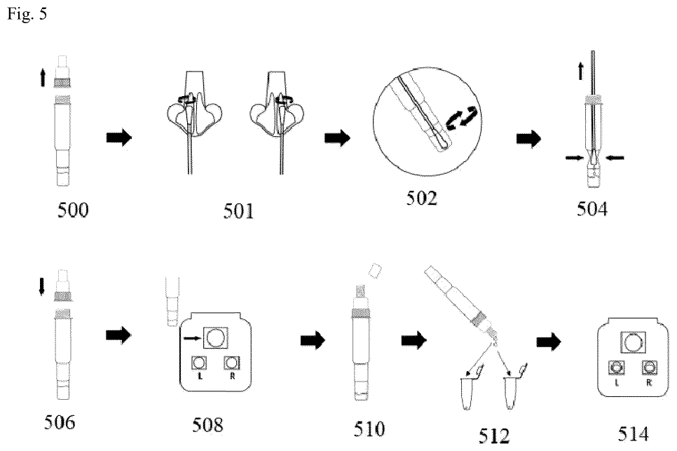

The collected biological sample is transferred into a sample extraction tube having a treatment buffer. When the collected biological sample is a nasal swab, nasopharyngeal swab, buccal swab, throat swab, urogenital or anal swab, or blood from a glass rod, the tip of the swab or glass rod having the collected biological sample is inserted into the treatment buffer and mixed (as shown in 402 of FIG. 4 and 502 of FIG. 5). The transferring further includes removing the swab or glass rode, where the sample extraction tube is squeezed to contact the swab to assist the collected biological sample on the swab or glass rod to transfer to the treatment buffer (as shown in 404 of FIG. 4, and 504 of FIG. 5), and attaching the cap to the tube of the sample extraction tube (as shown in 406 of FIG. 4 and 506 of FIG. 5). When the collected biological sample is transferred directly into the treatment buffer a volume of 400-600 microliters (μl) for the treatment buffer may be used.

When the collected biological sample is a biological sample in VTM or UTM, the transferring may include pouring the collected biological sample into the sample extraction tube, and attaching the cap to the tube of the sample extraction tube (as shown in 406 of FIGS. 4 and 506 of FIG. 5). When the collected biological sample is in VTM or UTM, the treatment buffer is used in a volume equal to the volume of the collected biological sample in the VTM or UTM.

The treatment buffer is selected based on the biological sample. For example, when the collected biological sample is nasal or oral swab the treatment buffer may include 1 mM to 100 mM Tris-HCl (pH 7.0 to 9.0). The treatment buffer may further include 0.1 mM to 0.5 mM EDTA having pH 8.0.

For example, when the collected biological sample is a nasal or oral swab the treatment buffer may include from 0 to 10% Bovine Serum Albumin, from 0.4 mM to 0.5 mM EDTA of pH 8, from 48 mM to 80 mM Guanidine isothiocyanate, and from 8 to 10 mM Tris-HCl of pH 9.0.

For example, when the biologic specimen is a nasal or oral swab the treatment buffer may include 0.125 mM sodium citrate of pH 6.62, 1 mM TCEP (tris (2-carboxyethyl) phosphine) of pH 4.5, and 0.04 mg/mL PVSA.

For example, when the biological sample is a nasal swab the treatment buffer may include 0.25 mM Sodium Citrate of pH 6.7 and 2 mM TCEP-HCl.

When the biological sample is an oral fluid sample, such as the chew rope from pigs, the treatment buffer may include 2 mM DCTA of pH 8, from 2 to 4 mM DTPA of pH 8, 1 mM EDTA of pH 8, 5 mM Sodium Citrate of pH 6.5, 2 mM TCEP-HCl.

When the biological sample is a serum sample the treatment buffer may include 2 mM DCTA of pH 8, from 2 to 4 mM DTPA of pH 8, 1 mM EDTA of pH 8, 1 mM EGTA of pH 8, 10 mM Tris HCl of pH 9.0.

When the biological sample is a processing fluid from pigs, the treatment buffer may include 3 mM magnesium chloride, 75 mM potassium chloride, 50 mM Tris HCl of pH 9.0, and from 0.25 mM to 5.0 mM TCEP, where the buffer is adjusted to pH 8.3, and combinations thereof.

In 304, the collected biological sample in the sample extraction tube is heated. The heating may include heating the transferred, collected biological sample contacted with the treatment buffer at from 80 to 95 degrees Celsius for from 2 to 10 minutes in the sample extraction tube.

The heating causes cell or protein capsule lysis to further release the nucleic acids into the treatment buffer. The heating may be conducted in a limited well thermal cycler, such as that described in PCT/US21/64256 titled LIMITED WELL THERMAL CYCLING DEVICE (referred to herein as limited well thermal cycling device) (as shown as 508 in FIG. 5). The heating may be conducted in a conventional heating block (as shown as 408 in FIG. 4).

In 306, after heating, the collected biological sample is dispensed into one or more reaction vessels. The dispensing includes removing the dropper cap of the sample extraction tube and dispensing the heated collected biological sample by squeezing the tube causing the collected biological sample to exit the dropper opening into a reaction vessel, such as a PCR tube or other conventional plastic tube (as shown by 410 and 412 in FIG. 4 and 510 and 512 in FIG. 5). The reaction vessel contains lyophilized reagents configured for carrying out an amplification reaction, such as polymerase chain reaction (PCR), reverse transcriptase (RT) PCR, real-time PCR, quantitative PCR, isothermal amplification on the collected biological sample. The reaction vessel may contain liquid reagents for carrying out an amplification reaction.

In 308, the collected biological sample contacted with the amplification reagents is analyzed for the presence or absence of microbial, viral, human, or animal (i.e. organisms) nucleic acid detection using conventional nucleic acid detection and identification methods, such as polymerase chain reaction (PCR), reverse transcriptase (RT) PCR, real-time PCR, quantitative PCR, isothermal amplification. For example, combined sequence amplification and nucleotide detection using the padlock probe as described in U.S. patent application Ser. No. 16/642,308 titled REACTION CONDITIONS COMPOSITION FOR CIRCULARIZING OGLIGONUCELEOTIDE PROBES (referred to herein as a C-SAND® analysis) may be used. For example, when the analysis is real-time PCR with florescence detection or C-SAND analysis with fluorescence detection the device to carry out the analysis may be the device as described in U.S. Pat. Nos. 9,568,429 and 9,810,631 titled WAVELENGTH SCANNING APPARATUS AND METHOD OF USE THEREOF. Or for example, analyzing may include conducting real-time PCR with fluorescence detection using the limited well thermal cycling device (as shown as 514 in FIG. 5). Or for example, the analysis may utilize conventional amplification devices. The analyzing further includes reporting the results of the presence or absence of the nucleic acid for detection.

The analysis step may optionally include a centrifuge step prior to nucleic acid detection that simultaneously separates non-cellular particulate matter and cellular debris from the nucleic acids for detection, when the biological sample includes non-cellular particulate matter, such as a chew rope from pigs or an oral fluid sample.

FIG. 4 represents a pictorial representation of the method 300 using a conventional PCR device. FIG. 4 also pictorially represents pre-method activities of removing the cap from the sample extraction tube 400 and collecting the biological sample through a nasal swab 401, resulting in the collected biological sample.

FIG. 5 represents a pictorial representation of the method 300 using a limited well thermal cycling device. FIG. 5 also pictorially represents pre-method activities of removing the cap from the sample extraction tube 500 and collecting the biological sample through a nasal swab 501, resulting in the collected biological sample.

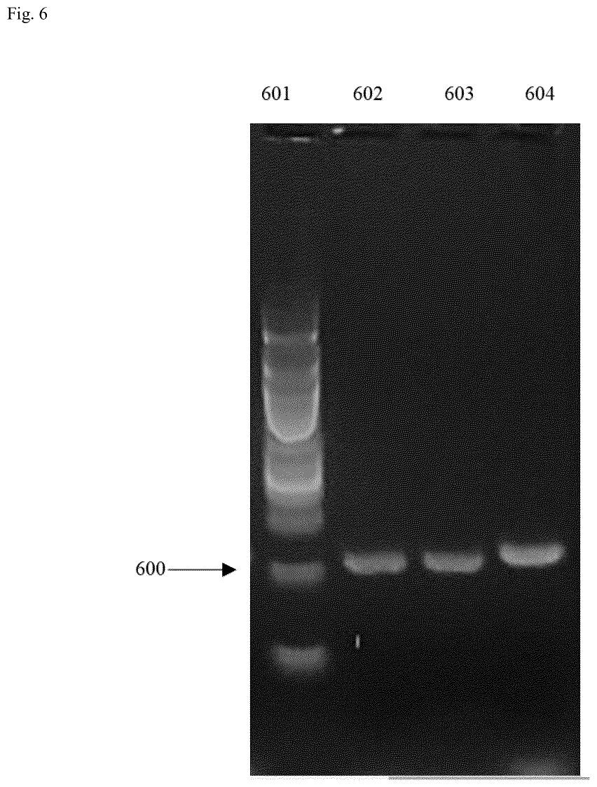

FIG. 6 demonstrates the efficacy of the method 300 of utilizing a sample extraction tube for nucleic acid detection, namely human DNA, without isolation or purification of nucleic acids and without utilizing specialized equipment in the preparation of the collected biological sample. In this example, the nucleic acids for detection were the HFE gene, the gene that causes hereditary hemochromatosis (HH) in humans and the human HBB (hemoglobin subunit beta) gene. Two sets of primers for the HFE gene detection were used. The first set of primers for the HFE gene amplification were HFE GtoA-F new (identified as SEQ ID NO: 1): 5′CCATGAAGTGGCTGAAGG3′ and HFE GtoA-R new (identified as SEQ ID NO: 2): 5′CTCAGCTCCTGGCTCTCATC3′, with the product 216 base pairs in length. The second set of primers for the HFE gene amplification were HFE-CtoG-F (identified as SEQ ID NO: 3):5′GTCTCCAGGTTCACACTCTC3′ and HFE-CtoG-R (identified as SEQ ID NO: 4): 5′GTGATCCCACCCTTTCAGACTC3′, with the product 220 base pairs in length. One set of primers was used for HBB detection. This set of primers for HBB amplification was SC-F new (identified as SEQ ID NO: 5): 5′GGCAGAGCCATCTATTGCTTAC3′ and SC-R new (identified as SEQ ID NO: 6): 5′CTCTGTCTCCACATGCCCAGTTTC3′, with this product 227 base pairs in length.

The method 300 was utilized in detecting the HFE and HBB genes. In particular, the collected biological sample is a nasal swab that was transferred into a sample extraction tube containing 0.6 mL of a treatment buffer of 0.025 mM Sodium Citrate of pH 6.62, 0.2 mM TCEP (tris(2-carboxyethyl)phosphine) of pH 4.5 and 0.008 mg/mL polyvinyl sulfonic acid (PVSA) using the procedure of 302. While this treatment buffer was used in this example, other treatment buffers may be used. The collected biological sample was heated at 95 degrees Celsius for 3 minutes.

The collected biological sample was then dispensed to a PCR tube using the procedure of 306, where 1 drop (approximately 35 μl) of the collected biological sample was dispensed to the PCR tube containing 15 μl of PCR reagents to create a final 50 μl reaction containing 10 mM Tris-HCl, 50 mM KCl, 1.5 mM MgCl₂ (pH8.3), 400 microMolar (μM) of all 4 deoxyribonucleotide triphosphate (dNTPs), 20 μM each of Forward and Reverse Primers, and 5 units (where units are defined the manufacturer and is a measure of the amount of enzyme that incorporates a molar concentration of dNTPs into perceptible DNA) the of Taq Polymerase. The PCR amplifications were done on a conventional PCR device, namely, the Life Tech (ABI 2720) Thermal Cycler device under the following cycling conditions: Initial heating at 95° C. for 3 minutes followed by 35 cycles of 94° C. for 30 seconds, 55° C. for 30 seconds, and 72° C. for 1 minute. After 35 cycles were completed, the reaction was held at 72° C. for 5 minutes. Twenty microliters of the PCR reaction was loaded on a 2% Agarose gel along with a 100 bp ladder marker, with 600 identifying the 200 base pair marker. The agarose gel is shown as FIG. 6, where the extreme left lane is a Molecular weight standard labeled as 601. The next two lanes show the two products from the HFE gene amplified with primers as described above, 602 (216 base pair product) and 603 (220 base pair product), respectively. The next lane to the right shows the expected PCR product from the human HBB gene labeled as 604 (227 base pair product). This data demonstrates that the method of direct nucleic acid detection using a sample extraction tube without isolation or purification of the nucleic acids and without specialized equipment in the sample preparation and delivery for PCR amplification isolates sufficient quality DNA that can be amplified with an amplification reaction. More particularly, the method 300 can be used to amplify sufficient quality DNA directly from the collected biological sample in a sample extraction tube.

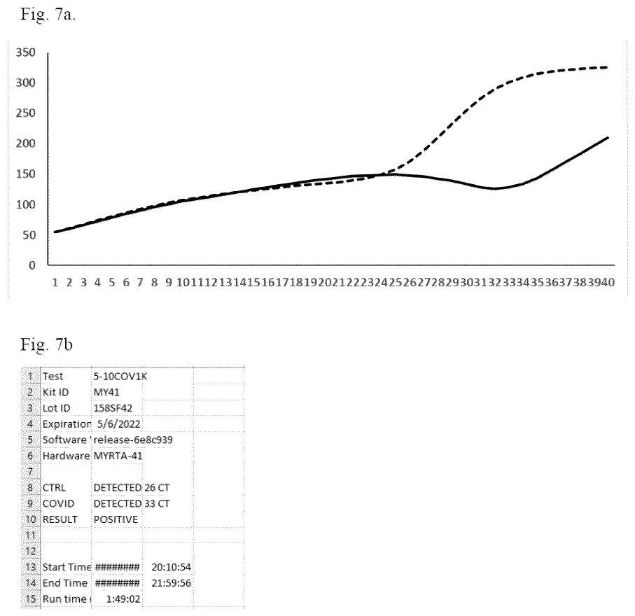

FIG. 7 demonstrates the efficacy of the method 300 of utilizing a sample extraction tube for direct nucleic acid detection, namely RNA detection, without isolation or purification of nucleic acids and without utilizing specialized equipment in the preparation of the collected biological sample. In this example, the nucleic acids for detection were the SARS-COV2 virus. The primer utilized in connection with the RNA amplification is complimentary to a portion of the RdRp gene of SARS-COV2.

The method 300 was utilized in detecting SARS-COV2. In particular, the collected biological sample is a nasal swab that was transferred into a sample extraction tube containing 0.6 mL of a treatment buffer of 0.125 mM sodium citrate of pH 6.62, 1 mM TCEP (tris(2-carboxyethyl)phosphine) of pH 4.5, and 0.04 mg/mL PVSA (polyvinyl sulfonic acid). While this treatment buffer was used in this example, other treatment buffers may be used. The collected biological sample, was heated at 95 degrees Celsius for 3 minutes using a limited well thermal cycling device.

The collected biological sample was then dispensed to a first PCR tube, where 4 drops (approximately 100 microliters) of the collected biological sample were added to a first PCR tube (left side of the limited well thermal cycling device as shown in 508 of FIG. 5) having all the PCR reagents necessary to amplify the RdRp gene of SARS-COV2. In particular, the TaqMan probe is labeled with the 6-FAM dye (absorbance max at 495 nm and emission max at 520 nm). This probe is specific to the PCR product amplified from the RdRp gene of the coronavirus.

Additionally, a control was used to validate the collected biological sample, where 4 drops (approximately 100 microliters) of the collected biological sample were added to a second PCR tube (right side of the device as shown in 508 of FIG. 5) having all the PCR reagents necessary to amplify the Cytochrome c Oxidase 1 (Cox-1) gene of the human. In particular, the TaqMan probe is labeled with Cy5 dye (absorbance max at 651 nanometers (nm) and emission at 670 nm). This particular probe will detect the specific region of the Cox-1 gene being amplified from the human cells collected from the nostrils and acts as an internal positive control.

Each of the PCR reactions described above is a 100 microliter reaction with lyophilized reagents that after rehydration with the collected biological sample contain 50 mM Tris-HCl (pH 8.3), 75 mM KCl, 3 mM MgCl₂, 10 mM DTT, 400 M of each dNTPs, 0.6 μM of each PCR primer, 0.2 μM of TaqMan probe, 10 Units of Taq DNA Polymerase, and 80 Units (where units are defined the manufacturer and is a measure of the amount of enzyme that incorporates a molar concentration of dNTPs into perceptible DNA) of MMLV Reverse Transcriptase. The PCR tubes are placed in the limited well thermal cycling device, where the device performed a set cycling condition which includes an initial incubation for 5 minutes at 42 degrees Celsius (° C.), followed by 94° C. for 2 min, followed by 40 cycles between 94° C. for 10 seconds and 54° C. for 1 minute.

The raw data from the limited well thermal cycling device is shown in FIG. 7a, which indicates that SARS-COV2 and Cox-1 were detected in the collected biological sample and is a graphical representation of the fluorescence detection. The X axis shows the number of cycles (Ct value) and the Y axis shows relative fluorescence expressed as arbitrary fluorescence units (AFUs). The dotted line shows fluorescence from the internal positive control (Cox-1) and the solid lines shows fluorescence from SARS-COV2 detection. FIG. 7b is the reported results of the analysis, where the Ct value for the positive control (Ct is 26 for Cox-1) and the test (Ct of 33 for Covid) are also indicated. This demonstrates that PCR amplification, including real-time PCR amplification, is possible from the sample collected and processed in a sample extraction tube according to the method 300.

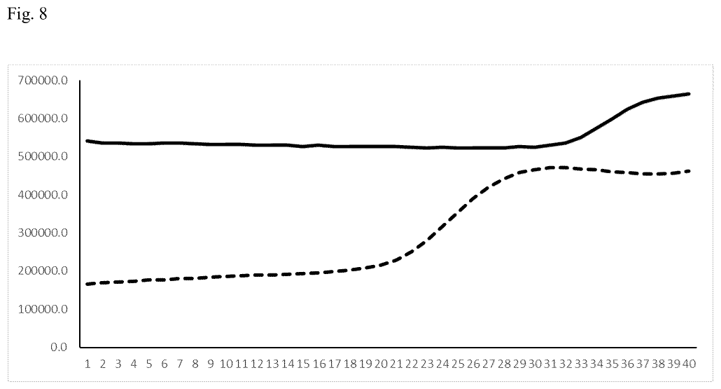

FIG. 8 demonstrates the efficacy of the method 300 of utilizing a sample extraction tube for direct nucleic acid detection, namely RNA detection, without isolation or purification of nucleic acids and without utilizing specialized equipment. In this example, the nucleic acids for detection were the SARS-COV2 virus. The primer utilized in connection with the RNA amplification is complimentary to a portion of the RdRp gene of SARS-COV2.

The method 300 was utilized in detecting SARS-COV2. In particular, the collected biological sample is a nasal swab that was transferred into a sample extraction tube containing 0.6 mL of a treatment buffer of 0.125 mM sodium citrate of pH 6.62, 1 mM TCEP (tris (2-carboxyethyl) phosphine) of pH 4.5, and 0.04 mg/mL PVSA (polyvinyl sulfonic acid). While this treatment buffer was used in this example, other treatment buffers may be used. The collected biological sample, was heated at 95 degrees Celsius for 3 minutes using a conventional heating block.

The collected biological sample was then dispensed to a first PCR tube, where 2 drops (approximately 50 microliters) of the collected biological sample were added to a first PCR tube having lyophilized PCR reagents necessary to amplify the RdRp gene of SARS-COV2. In particular, the TaqMan probe is labeled with the 6-FAM dye (absorbance max at 495 nm and emission max at 520 nm). This probe is specific to the PCR product amplified from the RdRp gene of the coronavirus. After the collected biological sample was dispensed, the first PCR tube contained, in addition to the collected biological sample, 50 milliMolar (mM) Tris-HCl (pH 8.3), 75 mM KCl, 3 mM MgCl₂, 10 mM DTT, 400 μM of each dNTPs, 0.6 μM of each PCR primer, 0.2 μM of TaqMan probe, 10 Units of Taq DNA Polymerase, and 80 Units of MMLV Reverse Transcriptase.

Additionally, a control was used to validate the collected biological sample, where 2 drops (approximately 50 microliters) of the collected biological sample were added to a second PCR tube having all the PCR reagents necessary to amplify the Cytochrome c Oxidase 1 (Cox-1) gene of the human. In particular, the TaqMan probe is labeled with Cy5 dye (absorbance max at 651 nm and emission at 670 nm). This particular probe will detect the specific region of the Cox-1 gene being amplified from the human cells collected from the nostrils and acts as an internal positive control. After the collected biological sample was dispensed, the first PCR tube contained, in addition to the collected biological sample, 50 mM Tris-HCl (pH 8.3), 75 mM KCl, 3 mM MgCl₂, 10 mM DTT, 400 μM of each dNTPs, 0.6 μM of each PCR primer, 0.2 μM of TaqMan probe, 10 Units of Taq DNA Polymerase, and 80 Units of MMLV Reverse Transcriptase. The PCR tubes are placed in conventional real-time PCR system, namely, an Applied Biosystems QuantStudio™ 5 Real-Time PRC System.

The System was set to run for 5 minutes at 42° C. followed by 94° C. for 2 minutes. This initial step was followed by 40 cycles at 94° C. for 10 seconds and 54° C. for 1 minute. The raw data curve of the data from the machine is shown in FIG. 8. In FIG. 8, the X axis shows the number of cycles and the Y axis shows relative fluorescence presented as arbitrary fluorescence unit (AFUs). Data shows that for the first PCR tube the System detected the presence of SARS-CoV2 at a Ct value of 33 (solid line), and for the second PCR tube the System detected the presence of the internal control (Cox-1) at a Ct value of 24 (dotted line). This demonstrates that PCR amplification of RNA, including real-time PCR amplification, is possible from the sample collected and processed in a sample extraction tube according to the method 300.

1. A method of direct human, animal, microbial, and viral nucleic acid detection from a collected biological sample using a sample extraction tube without isolation or purification of the nucleic acids and without the use of specialized equipment in the collected biological sample preparation, the method comprising:
transferring the collected biological sample to a sample extraction tube having a treatment buffer configured for stabilizing the nucleic acids of the collected biological sample, the sample extraction tube comprising
a tube configured for accommodating a volume of liquid from 0.5 to 5 milliliters, the tube made of a flexible plastic material;
a cap configured for removable attachment to the tube, the cap made of a plastic material that is rigid, wherein the cap has a dropper opening and a dropper cap;
heating the collected biological sample transferred to the sample extraction tube from 2 to 10 minutes at from 80 to 95 degrees Celsius;
dispensing the collected biological sample from the dropper opening of the sample extraction tube to at least one reaction vessel;
analyzing the collected biological sample for the nucleic acid;
reporting results of the analysis to determine the presence or absence of the nucleic acid.

2. The method of claim 1, wherein
the nucleic acid analyzed is a nucleic acid of an organism's genome.

3. The method of claim 1, wherein
the nucleic acid analyzed is a nucleic acid from a pathogen.

4. The method of claim 1, wherein
the analyzing is a nucleic acid amplification reaction selected from the group consisting of polymerase chain reaction (PCR), reverse transcriptase (RT) PCR, real-time PCR, real-time quantitative PCR, and isothermal amplification.

5. The method of claim 1, wherein
the collected biological sample is a nasal swab from a human, where the nucleic acid analyzed detects the presence of any RNA or DNA virus.

6. The method of claim 5, wherein
the collected biological sample is a nasal swab from a human, where the nucleic acid analyzed is a RdRp gene of SARS-COV2;
the treatment buffer is 0.125 mM sodium citrate of pH 6.62, 1 mM TCEP (tris(2-carboxyethyl)phosphine) of pH 4.5, and 0.04 mg/mL PVSA (polyvinyl sulfonic acid), and wherein
the analysis is real-time polymerase chain reaction with fluorescence detection.

7. The method of claim 1, wherein
the collected biological sample is a nasal swab from a human, where the nucleic acid analyzed detects the presences of nucleic acids that cause hereditary genetic conditions in humans.

8. The method of claim 7, wherein
the treatment buffer is 0.025 mM Sodium Citrate of pH 6.62, 0.2 mM TCEP (tris (2-carboxyethyl) phosphine) of pH 4.5, and 0.008 mg/mL polyvinyl sulfonic acid (PVSA), and wherein
the analysis is polymerase chain reaction with agarose gel electrophoresis.

9. The method of claim 7, wherein
the analysis is real-time polymerase chain reaction with fluorescence detection.

10. The method of claim 7, wherein
the treatment buffer is 0.025 mM Sodium Citrate of pH 6.62, 0.2 mM TCEP (tris (2-carboxyethyl) phosphine) of pH 4.5, and 0.008 mg/mL polyvinyl sulfonic acid (PVSA), and wherein
the analysis is isothermal amplification with fluorescence detection.

11. A method of direct human, animal, microbial, and viral nucleic acid detection from a collected biological sample using a sample extraction tube, the method comprising:
transferring the collected biological sample to the sample extraction tube without prior isolation or purification of nucleic acids having a treatment buffer configured for stabilizing the nucleic acids of the collected biological sample, the sample extraction tube comprising
a tube configured for accommodating a volume of liquid from 0.5 to 5 milliliters, the tube made of a flexible plastic material;
a cap configured for removable attachment to the tube, the cap made of a plastic material that is rigid, wherein the cap has a dropper opening;
heating the collected biological sample transferred to the sample extraction tube from 2 to 10 minutes at from 80 to 95 degrees Celsius;
dispensing the collected biological sample from the dropper opening of the sample extraction tube to at least one reaction vessel without the use of specialized equipment;
analyzing the collected biological sample for the nucleic acid without isolation or purification of the nucleic acids prior to the analyzing;
reporting results of the analysis to determine the presence or absence of the nucleic acid.

12. The method of claim 11, wherein
the sample extraction tube further comprises a dropper cap.

13. The method of claim 11, wherein
the treatment buffer comprises a buffering agent.

14. The method of claim 13, wherein
the buffering agent is selected from the group consisting of phosphate buffered saline, from 5 to 50 milli-Molar (mM) Tris HCl, from 0.05-0.5 mM sodium citrate of pH from 6.0 to 7.0, 3 mM magnesium chloride, and 75 mM potassium chloride, and combinations thereof.

15. The method of claim 13, wherein
the treatment buffer further comprises a chelating agent that stabilizes the released nucleic acids of the biological sample and interacts with other cellular components and cellular debris contained in the sample to facilitate analyzing the nucleic acids without isolation or purification of the nucleic acids.

16. The method of claim 15, wherein
the buffering agent is selected from the group consisting of phosphate buffered saline, from 5 to 50 milli-Molar (mM) Tris HCl, from 0.05-0.5 mM sodium citrate of pH from 6.0 to 7.0, 3 mM magnesium chloride, and 75 mM potassium chloride, and combinations thereof; and
the chelating agent is selected from the group consisting of from 0.1 to 1 milliMolar (mM) EDTA of pH 8.0, 2 mM DCTA of pH 8, from 0.5-4 mM DTPA of pH 8, from 0.25 to 5 mM TCEP-HCl of pH 4.5, 1 mM EGTA, and from 5% to 10% (weight/volume) of Bovine Serum Albumin, and combinations thereof.

17. The method of claim 15, wherein
the treatment buffer further comprises a lysis agent that further facilitates lysis of cellular membranes, nuclei, and protein coats.

18. The method of claim 17, wherein
the buffering agent is selected from the group consisting of phosphate buffered saline, from 5 to 50 milli-Molar (mM) Tris HCl, from 0.05-0.5 mM sodium citrate of pH from 6.0 to 7.0, 3 mM magnesium chloride, and 75 mM potassium chloride, and combinations thereof;
the chelating agent is selected from the group consisting of from 0.1 to 1 milliMolar (mM) EDTA of pH 8.0, 2 mM DCTA of pH 8, from 0.5-4 mM DTPA of pH 8, from 0.25 to 5 mM TCEP-HCl pH 4.5, 1 mM EGTA, and from 5% to 10% (weight/volume) of Bovine Serum Albumin, and combinations thereof; and
the lysis agent is selected from the group consisting of from 20 to 250 mM guanidine isothicynate and 2 mM TCEP-HCl, and combinations thereof.

19. The method of claim 17, wherein
the treatment buffer further includes an RNase inhibitor.


SEQUENCE LISTING (6 sequences)

SEQ ID NO: 1 - DNA, 18 bp, Homo sapiens
  source 1..18: mol_type = other DNA
ccatgaagtggctgaagg

SEQ ID NO: 2 - DNA, 20 bp, Homo sapiens
  source 1..20: mol_type = other DNA
ctcagctcctggctctcatc

SEQ ID NO: 3 - DNA, 20 bp, Homo sapiens
  source 1..20: mol_type = other DNA
gtctccaggttcacactctc

SEQ ID NO: 4 - DNA, 22 bp, Homo sapiens
  source 1..22: mol_type = other DNA
gtgatcccaccctttcagactc

SEQ ID NO: 5 - DNA, 22 bp, Homo sapiens
  source 1..22: mol_type = other DNA
ggcagagccatctattgcttac

SEQ ID NO: 6 - DNA, 24 bp, Homo sapiens
  source 1..24: mol_type = other DNA
ctctgtctccacatgcccagtttc